(19) United States
(12) Patent Application Publication
(10) Pub. No.: US 2025/0221878 A1
(43) Pub. Date: Jul. 10, 2025

(54) WEARABLE DEVICE OPERATION METHOD FOR CONTROLLING GENERATION OF EXTERNAL FORCE, AND WEARABLE DEVICE

(71) Applicant: Samsung Electronics Co., Ltd., Suwon-si (KR)

(72) Inventors: Sungmin SHIN, Suwon-si (KR); Donghee KANG, Suwon-si (KR); Seungjoon LEE, Suwon-si (KR)

(21) Appl. No.: 19/090,088

(22) Filed: Mar. 25, 2025

Related U.S. Application Data
(63) Continuation of application No. PCT/KR2023/014018, filed on Sep. 18, 2023.

(30) Foreign Application Priority Data
Oct. 5, 2022 (KR) 10-2022-0126955

(51) Int. Cl. A61H 3/00; A61B 5/11; A61H 1/02

(52) U.S. Cl. CPC A61H 3/00; A61B 5/112; A61H 1/0244; A61H 2201/5007; A61H 2201/5048

(57) ABSTRACT

A wearable device operation method for controlling generation of external force, and/or a wearable device are disclosed. An operation method of a wearable device according to an example embodiment may include: on the basis of detection of a pattern change in sensor data and detection of an utterance trigger signal, detecting a target situation; and in response to the detection of the target situation, switching an operation mode of the wearable device to an intervention blocking mode for stopping generation of external force.

### CROSS-REFERENCE TO RELATED APPLICATIONS

This application is a continuation application of International Application No. PCT/KR2023/014018 designating the United States, filed on Sep. 18, 2023, in the Korean Intellectual Property Receiving Office and claiming priority to Korean Patent Application No. 10-2022-0126955, filed on Oct. 5, 2022, the disclosures of which are all hereby incorporated by reference herein in their entireties.

### BACKGROUND

### Technical Field

Certain example embodiments may relate to an operation method of a wearable device for controlling the generation of external force, and/or the wearable device.

### Description of Related Art

A wearable computer or a wearable device may refer to an electronic device that may be worn, such as glasses, watches, clothing, an exercise device, a walking assist device, and/or any other suitable wearable device. The wearable computer or wearable device is being actively developed to supplement or enhance human abilities. A user may wear the device like a part of their body, for example without any discomfort. A wearable device may continuously and in real time collect detailed information on the surrounding environment or changes in an individual's body. For example, the wearable device may record information about the surroundings through various sensor(s), and/or may measure biosignals such as a heartbeat and information on the posture and position of the device. The wearable device may provide a user with various functions and information based on the information collected through the sensors.

### SUMMARY

A wearable device according to an example embodiment may include driving modules configured to generate external force corresponding to an operation mode. Each driving module may comprise a motor and/or circuitry. The wearable device may include a memory in which computer-executable instructions are stored. The wearable device may include at least one processor, comprising processing circuitry, individually and/or collectively configured to execute the instructions by accessing the memory. The instructions may be configured to cause the at least one processor to detect a target situation, based on detection of a pattern change in sensor data and detection of an utterance trigger signal. The instructions may be configured to cause the at least one processor to switch an operation mode of the wearable device to an intervention blocking mode to stop the generation of external force, in response to the detection of the target situation.

An operation method of the wearable device according to an example embodiment may include detecting a target situation, based on the detection of a pattern change in sensor data and/or the detection of an utterance trigger signal. The operation method of the wearable device may include switching the operation mode of the wearable device to an intervention blocking mode to stop the generation of external force, in response to the detection of the target situation.

### BRIEF DESCRIPTION OF DRAWINGS

FIG. 1 is a diagram illustrating an overview of a wearable device worn on a user's body according to an example embodiment.

FIG. 2 is a diagram illustrating an exercise management system including a wearable device and an electronic device, according to an example embodiment.

FIG. 3 is a front view of a wearable device according to an example embodiment.

FIG. 4 is a side view of a wearable device according to an example embodiment.

FIGS. 5A and 5B are diagrams illustrating a configuration of a control system of a wearable device according to an example embodiment.

FIG. 6 is a diagram illustrating an interaction between a wearable device and an electronic device according to an example embodiment.

FIG. 7 is a flowchart illustrating an operation of a wearable device according to an example embodiment.

FIG. 8 is a diagram illustrating a motion of a user wearing a wearable device over time according to an example embodiment.

FIGS. 9A to 9C are diagrams illustrating user interface (UI) screens for setting an intervention blocking mode of a program for controlling a wearable device according to an example embodiment.

FIG. 10 is a flowchart illustrating an operation of a wearable device corresponding to whether a target situation has been terminated after switching to an intervention blocking mode, according to an example embodiment.

### DETAILED DESCRIPTION

The following detailed structural or functional description is provided as an example only and various alterations and modifications may be made to embodiments. Accordingly, the embodiments are not construed as limited to the disclosure and should be understood to include all changes, equivalents, and replacements within the idea and the technical scope of the disclosure.

As used herein, the singular forms “a”, “an”, and “the” include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

Hereinafter, embodiments will be described in detail with reference to the accompanying drawings. When describing the embodiments with reference to the accompanying drawings, like reference numerals refer to like elements and a repeated description related thereto will be omitted.

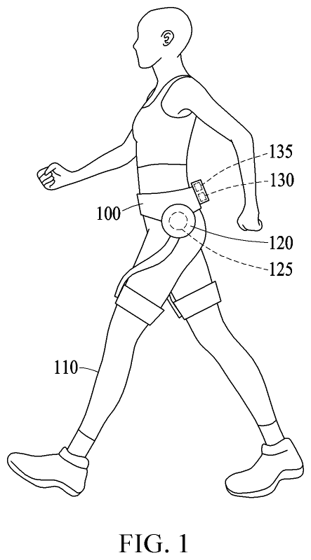

FIG. 1 is a diagram illustrating an overview of a wearable device worn on a user's body, according to an embodiment.

Referring to FIG. 1, a wearable device 100 may be a device worn on the body of a user 110 to assist the user 110 in walking, exercising, and/or working. The term “wearable device” may be replaced with “wearable robot,” “walking assistance device,” or “exercise assistance device.” The user 110 may be a human or an animal, but is not limited thereto. The wearable device 100 may be worn on a body (e.g., a lower body (the legs, ankles, knees, etc.), an upper body (the torso, arms, wrists, etc.), or the waist) of the user 110 to apply external force such as an assistance force and/or a resistance force to a body motion of the user 110. The assistance force may be a force applied in the same direction as the body motion direction of the user 110, the force to assist a body motion of the user 110. The resistance force may be a force impeding the body motion of the user 110, which is applied in an opposite direction to the direction of the body motion of the user 110. The term “resistance force” may also be referred to as an “exercise load”.

According to an embodiment, the wearable device 100 may generate an external force corresponding to a set operation mode. For example, an operation mode of the wearable device 100 may include, but is not limited to, a walking assistance mode, an exercise assistance mode, and an intervention blocking mode. The wearable device 100 may be set to an operation mode and switched to another operation mode. The operation mode of the wearable device 100 may be determined by a user input or may be automatically determined according to predetermined criteria.

In an embodiment, when the wearable device 100 operates in the walking assistance mode to assist the user 110 in walking, the wearable device 100 may assist the walking of the user 110 by applying an assistance force generated through a driving module 120 of the wearable device 100 to the body of the user 110. The wearable device 100 may enable the user 110 to walk independently or to walk for a long time by providing a force required for the user 110 to walk, thereby expanding the walking ability of the user 110. The wearable device 100 may also improve the walking of a user having an abnormal walking habit or posture.

In an embodiment, when the wearable device 100 operates in an exercise assistance mode to enhance the exercise effect of the user 110, the wearable device 100 may hinder a body motion of the user 110 or provide resistance to a body motion of the user 110 by applying a resistance force generated by the driving module 120 to the body of the user 110. When the wearable device 100 is a hip-type wearable device that is worn on the waist (or pelvis) and legs (e.g., thighs) of the user 110, the wearable device 100 may provide an exercise load to a body motion of the user 110 while being worn on the legs, thereby enhancing the exercise effect on the legs of the user 110. In an embodiment, in the exercise assistance mode, the wearable device 100 may apply an assistance force to the body of the user 110 to assist a body motion of the user 110. In an embodiment, in the exercise assistance mode, the wearable device 100 may provide a combination of an assistance force and a resistance force for each exercise session or time interval, for example, providing an assistance force in some exercise sessions and providing a resistance force in other exercise sessions.

In an embodiment, when the wearable device 100 operates in the intervention blocking mode to block intervention in the motions of the user 110, the wearable device 100 may stop the generation of an external force applied to the user. For example, in response to switching to the intervention blocking mode, any external force being applied to the user by the wearable device 100 may be immediately stopped. For example, in response to switching to the intervention blocking mode, the generation of external force may be gradually stopped such that the magnitude of the external force applied to the user from the wearable device 100 may be reduced by a certain amount at a certain period.

In embodiments of the present disclosure, for ease of description, the wearable device 100 is described as an example of a hip-type wearable device, as illustrated in FIG. 1, but the embodiments are not limited thereto. As described above, the wearable device 100 may be worn on body parts (e.g., upper arms, lower arms, hands, calves, and feet) other than the waist and legs (particularly, the thighs), and a shape and configuration of the wearable device 100 may vary depending on the body part on which the wearable device 100 is worn.

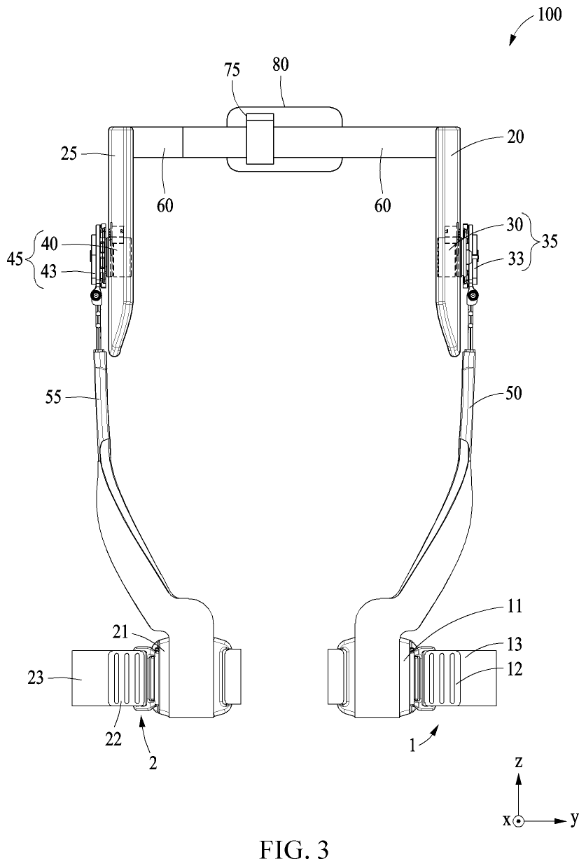

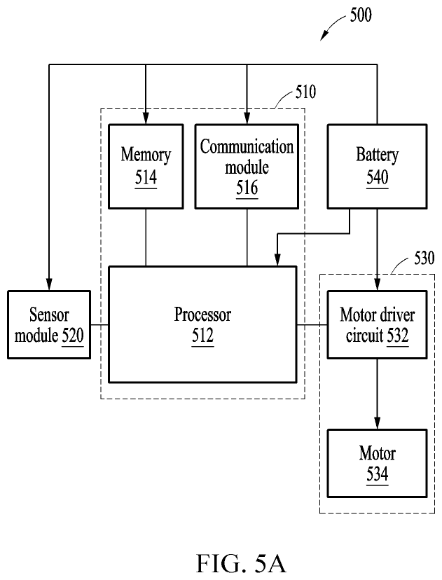

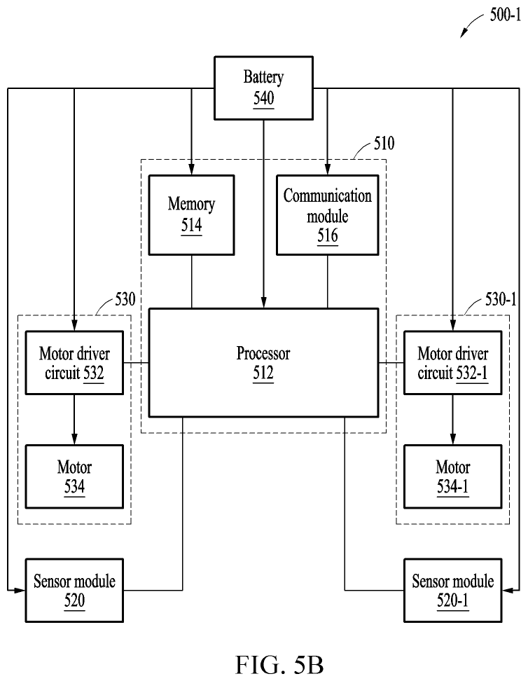

According to an embodiment, the wearable device 100 may include a support frame (e.g., leg support frames 50 and 55 and waist support frames 20 and 25 of 30 FIG. 3) configured to support the body of the user 110 when the wearable device 100 is worn on the body of the user 110, a sensor module (e.g., a sensor module 520 of FIG. 5A, comprising at least one sensor) configured to obtain sensor data including motion information on a body motion (e.g., a motion of a leg, and a motion of an upper body) of a user, the driving module 120 (e.g., driving modules 35 and 45 of FIG. 3) configured to generate an external force to be applied to a leg of a user, and a control module 130 (e.g., a control module 510 of FIGS. 5A and 5B) configured to control the wearable device 100.

The sensor module may include an angle sensor 125 and an inertial measurement unit (IMU) 135. The angle sensor 125 may measure a hip joint angle value of the user 110. The angle sensor 125 may include, for example, an encoder and/or a hall sensor. In an embodiment, the angle sensor 125 may be positioned near the left hip joint and the right hip joint and may measure a hip joint angle value of the left hip joint of the user 110 and a hip joint angle value of the right hip joint of the user 110, respectively. The hip joint angle value of the left hip joint may correspond to an angle of the left leg of the user 110, and the hip joint angle value of the right hip joint may correspond to an angle of the right leg of the user 110. The IMU 135 may measure a change in acceleration and rotation velocity according to the motion of the user 110. The IMU 135 may measure, for example, a motion value of the upper body of the user 110. The IMU 135 may include an acceleration sensor, and/or an angular velocity sensor.

In an embodiment, the control module 130 and the IMU 135 may be disposed in a housing (e.g., a housing 80 of FIG. 3) of the wearable device 100. The housing may be formed or attached to the outside of the support frame of the wearable device 100, and may be disposed behind the waist when the user 110 wears the wearable device 110, however, the embodiments are not limited thereto.

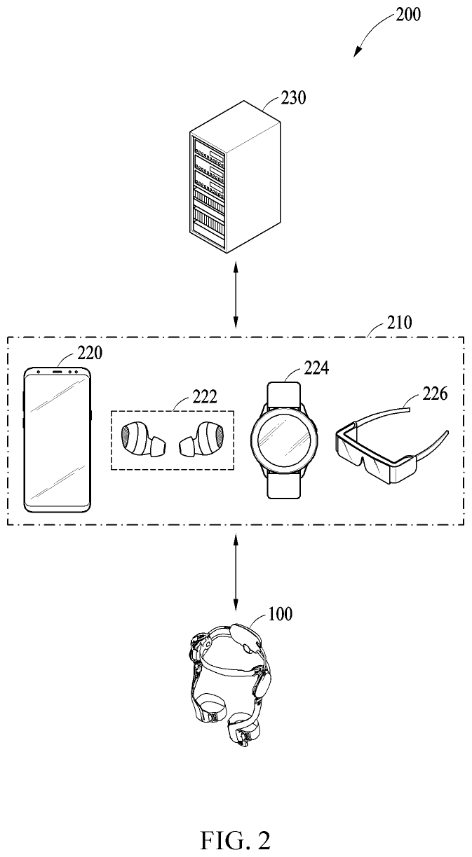

FIG. 2 is a diagram illustrating a management system including a wearable device and an electronic device according to an embodiment.

Referring to FIG. 2, a management system 200 may include the wearable device 100 (e.g., the wearable device 100 of FIG. 1) for assisting a body motion of a user, an electronic device 210, and a server 230. In an embodiment, at least one (e.g., the electronic device 210 or the server 230) of the above-described devices may be omitted from the management system 200, or one or more other devices (e.g., an exclusive controller device for the wearable device 100) may be added to the management system 200.

According to an embodiment, the wearable device 100 may interoperate with the electronic device 210. The electronic device 210 may be a device in various forms. The electronic device 210 may include, for example, a user terminal 220, wireless earphones 222, a smartwatch 224, or smart glasses 226, but are not limited thereto. The user terminal 220 may include, for example, a portable communication device (e.g., a smartphone, a tablet, a personal computer (PC)), a computer device, a portable multimedia device, or a home appliance, but is not limited thereto. In an embodiment, the wearable device 100 and the electronic device 210 may be connected to each other via wireless communication (e.g., Bluetooth™ communication).

According to an embodiment, a user (e.g., the user 110 of FIG. 1) may control an operation of the wearable device 100 through the electronic device 210. The electronic device 210 may communicate with the wearable device 100 and may remotely control the wearable device 100 or provide state information (e.g., remaining battery power) of the wearable device 100 to the user.

In an embodiment, the electronic device 210 may execute a program (e.g., an application) to control the wearable device 100, and the user may set an operation mode of the wearable device 100 or control an operation of the wearable device 100, such as control the power, through the program. The user may adjust a setting value (e.g., torque intensity or audio volume) of the wearable device 100 through the program.

According to an embodiment, when linked to the wearable device 100, the electronic device 210 may provide input/output interface functions of the wearable device 100. A signal generated from the wearable device 100 may be provided to the user through an output device such as a display or a speaker of the electronic device 210. An input signal received through an input device of the electronic device 210 may be provided to the wearable device 100 in the form of a command. The wearable device 100 may perform an operation corresponding to a received command.

In an embodiment, for example, the wearable device 100 may obtain sensor data from the electronic device 210. The sensor data may include, for example, sensor data related to a user's motions and data related to voice signals. The wearable device 100 may operate using the sensor data obtained from a sensor module of the wearable device 100 and the sensor data obtained from the electronic device 210.

According to an embodiment, the electronic device 210 may be connected to the server 230 using short-range wireless communication or cellular communication. The server 230 may receive user information (e.g., a name, an age, and gender) of a user using the wearable device 100 and/or data related to the user's motions from the electronic device 210 and may store and manage the received user information and/or data. The server 230 may provide a program for controlling the wearable device 100 to the electronic device 210. The wearable device 100 and the electronic device 210 may communicate with each other through the server 230.

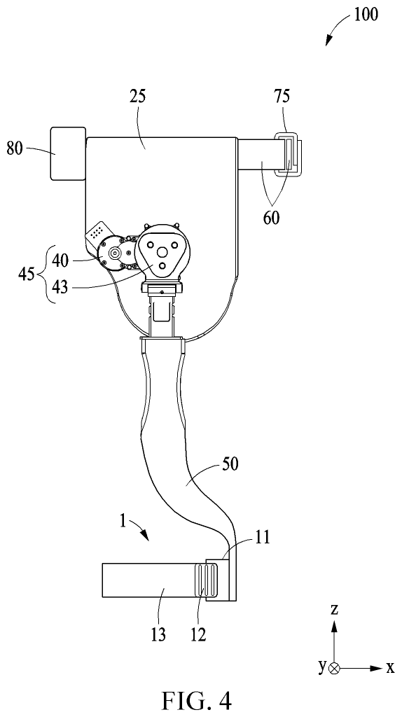

FIG. 3 is a front view of a wearable device according to an embodiment, and FIG. 4 is a side view of the wearable device according to an embodiment.

Referring to FIGS. 3 and 4, the wearable device 100 worn on the body of a user according to an embodiment may include the housing 80, the waist support frames 20 and 25, the driving modules 35 and 45, the leg support frames 50 and 55, thigh fastening portions 1 and 2, and a waist fastening portion. The waist fastening portion may include a belt 60 and an auxiliary belt 75. In an embodiment, at least one (e.g., the auxiliary belt 75) of the above-described components may be omitted from the wearable device 100, or one or more other components (e.g., the thigh fastening portions 1 and 2, or a fastening detection module to detect the fastening of the waist fastening portion) may be added to the wearable device 100.

In an embodiment, a control module (not shown) (e.g., the control module 130 of FIG. 1 or the control module 510 of FIGS. 5A and 5B), an IMU (not shown) (e.g., the IMU 135 of FIG. 1), and a battery (not shown) may be disposed in the housing 80. The housing 80 may protect the control module, the IMU, and the battery. For example, the housing 80 may be disposed on the back or a rear side of the waist of a user based on a state in which the wearable device 100 is worn on the body of the user. The control module may generate a control signal to control the wearable device 100 and may control an operation of the wearable device 100 based on the control signal. The control module may include a control circuit, which includes a processor configured to control actuators 30 and 40 of the driving modules 35 and 45, a memory, and a communication module comprising communication circuitry. The control module may further include a power supply module (not shown) to supply power from a battery to each of the components of the wearable device 100.

In an embodiment, the wearable device 100 may include a sensor module (not shown) (e.g., the sensor module 520 of FIG. 5A) configured to obtain sensor data from at least one sensor. The sensor module may obtain sensor data that changes according to a motion of a user. The sensor module is described in detail below.

In an embodiment, the waist support frames 20 and 25 may support a body part of the user when the wearable device 100 is worn on the body of the user. The waist support frames 20 and 25 may contact at least a portion of the outer surface of the user. The waist support frames 20 and 25 may be bent in a shape corresponding to a portion in contact with the body of the user. For example, the waist support frames 20 and 25 may have a shape surrounding the outer surface of the waist (or pelvis) of the user and may support the waist or pelvis of the user. The waist support frames 20 and 25 may include the first waist support frame 25 to support the right side of the user's waist, and the second waist support frame 20 to support the left side of the user's waist. The waist support frames 20 and 25 may be connected, directly or indirectly, to the housing 80.

The waist fastening portion may be connected, directly or indirectly, to the waist support frames 20 and 25 to fix the waist support frames 20 and 25 to the user's waist. The waist fastening portion may include, for example, a pair of belts 60 and the auxiliary belt 75. The auxiliary belt 75 may be connected, directly or indirectly, to one of the pair of belts 60.

In an embodiment, the pair of belts 60 may be connected, directly or indirectly, to the waist support frames 20 and 25. In a state before the user wears the wearable device 100, the pair of belts 60 may maintain a shape extending in a front direction (e.g., a +x direction) and may not impede the user from entering inside the pair of waist support frames 20 and 25. When the user enters inside the pair of waist support frames 20 and 25, the pair of belts 60 may be deformed and may surround a front portion of the user. The waist support frames 20 and 25 and the pair of belts 60 may entirely surround the circumference of the user's waist. In an embodiment, the auxiliary belt 75 may fasten the pair of belts 60 to each other while the pair of belts 60 overlap with each other. For example, one of the pair of belts 60 may wrap the other belt together with the auxiliary belt 75.

The driving modules 35 and 45 may generate an external force (or torque) to be applied to the body of the user based on a control signal generated by the control module. For example, the driving modules 35 and 45 may generate an external force to be applied to the user's legs under the control of the control module. In an embodiment, the driving modules 35 and 45 may include the first driving module 45 disposed at a position corresponding to the right hip joint of the user and the second driving module 35 disposed at a position corresponding to the left hip joint of the user. The first driving module 45 may include the first actuator 40 and a first joint member 43, and the second driving module 35 may include the second actuator 30 and a second joint member 33. The first actuator 40 may provide power to be transmitted to the first joint member 43 and the second actuator 30 may provide power to be transmitted to the second joint member 33. The first actuator 40 and the second actuator 30 may each include a motor configured to generate power (or torque) by receiving power from a battery. When the motor is supplied with electric power and driven, the motor may provide a force (an assistance force) to assist a body motion of the user or a force (a resistance force) to hinder a body motion of the user. In an embodiment, the control module may adjust the strength and direction of the force generated by the motor by adjusting a voltage and/or a current supplied to the motor.

In an embodiment, the first joint member 43 and the second joint member 33 may receive power from the first actuator 40 and the second actuator 30, respectively, and may apply external force to the body of the user based on the received power. The first joint member 43 and the second joint member 33 may be disposed at positions corresponding to the joint portions of the user, respectively. The first joint member 43 and the second joint member 33 may be disposed on one side of the waist support frames 25 and 20, respectively. One side of the first joint member 43 may be connected, directly or indirectly, to the first actuator 40, and the other side of the first joint member 43 may be connected, directly or indirectly, to the first leg support frame 55. The first joint member 43 may be rotated by the power received from the first actuator 40. An encoder or a hall sensor that may operate as an angle sensor to measure a rotation angle (corresponding to a joint angle of the user) of the first joint member 43 may be disposed on one side of the first joint member 43. One side of the second joint member 33 may be connected, directly or indirectly, to the second actuator 30, and the other side of the second joint member 33 may be connected, directly or indirectly, to the second leg support frame 50. The second joint member 33 may be rotated by the power received from the second actuator 30. An encoder or a hall sensor that may operate as an angle sensor to measure a rotation angle of the second joint member 33 may be disposed on one side of the second joint member 33.

In an embodiment, the first actuator 40 may be disposed in a lateral direction of the first joint member 43, and the second actuator 30 may be disposed in a lateral direction of the second joint member 33. A rotation axis of the first actuator 40 and a rotation axis of the first joint member 43 may be spaced apart from each other, and a rotation axis of the second actuator 30 and a rotation axis of the second joint member 33 may also be spaced apart from each other. However, embodiments are not limited thereto, and the actuators 30 and 40 and the joint members 33 and 43 may share a rotation axis. In an embodiment, the actuators 30 and 40 may be spaced apart from the joint members 33 and 43, respectively. In this case, the driving modules 35 and 45 may further include a power transmission module (not shown) configured to transmit power from the actuators 30 and 40 to the joint members 33 and 43. The power transmission module may be a rotary body, such as a gear, or a longitudinal member, such as a wire, a cable, a string, a spring, a belt, or a chain. However, the scope of the embodiment is not limited by the positional relationship between the actuators 30 and 40 and the joint members 33 and 43 and the power transmission structure described above.

In an embodiment, the leg support frames 50 and 55 may support a leg (e.g., a thigh) of the user when the wearable device 100 is worn on the leg of the user. For example, the leg support frames 50 and 55 may transmit the power generated by the driving modules 35 and 45 to the thighs of the user, and the power may act as an external force to be applied to the motion of the user's legs. One end of the leg support frames 50 and 55 may be connected, directly or indirectly, to the joint members 33 and 43 to rotate, and as the other end of the leg support frames 50 and 55 is connected, directly or indirectly, to covers 11 and 21 of the thigh fastening portions 1 and 2, the leg support frames 50 and 55 may transmit the power generated by the driving modules 35 and 45 to the thighs of the user while supporting the thighs of the user. For example, the leg support frames 50 and 55 may push or pull the thighs of the user. The leg support frames 50 and 55 may extend in a longitudinal direction of the thighs of the user. The leg support frames 50 and 55 may be bent to surround at least a portion of the circumference of the thighs of the user. For example, an upper portion of the leg support frames 50 and 55 may cover a portion oriented in a lateral direction (e.g., a +y direction or a −y direction) of the user's body, and a lower portion of the leg support frames 50 and 55 may cover a portion oriented in a front direction (e.g., the +x direction) of the user's body. The leg support frames 50 and 55 may include the first leg support frame 55 to support the right leg of the user, and the second leg support frame 50 to support the left leg of the user.

The thigh fastening portions 1 and 2 may be connected, directly or indirectly, to the leg support frames 50 and 55 and may fix the leg support frames 50 and 55 to the thighs. The thigh fastening portions 1 and 2 may include the first thigh fastening portion 2 to fix the first leg support frame 55 to the right thigh of the user, and the second thigh fastening portion 1 to fix the second leg support frame 50 to the left thigh of the user. The first thigh fastening portion 2 may include the first cover 21, a first fastening frame 22, and a first strap 23, and the second thigh fastening portion 1 may include the second cover 11, a second fastening frame 12, and a second strap 13.

In an embodiment, the covers 11 and 21 may apply torque generated by the driving modules 35 and 45 to the thighs of the user. For example, the covers 11 and 21 may be disposed on one side of a thigh of the user to push or pull the thigh of the user. The covers 11 and 21 may be disposed on a front surface of the thighs of the user. The covers 11 and 21 may be disposed in a circumferential direction of the thighs of the user. The covers 11 and 21 may extend to both sides from the other end of the leg support frames 50 and 55 and may include a curved surface corresponding to the thighs of the user. One end of the covers 11 and 21 may be connected to the fastening frames 12 and 22 and the other end of the covers 11 and 21 may be connected to the straps 13 and 23.

In an embodiment, one end of the fastening frames 12 and 22 may be connected to one side of the covers 11 and 21 and the other end of the fastening frames 12 and 22 may be connected to the straps 13 and 23. For example, the fastening frames 12 and 22 may be disposed to enclose at least a portion of the circumference of the thighs of the user such that the thighs of the user may not be separated from the leg support frames 50 and 55. The first fastening frame 22 may have a fastening structure that connects the first cover 21 and the first strap 23, and the second fastening frame 12 may have a fastening structure that connects the second cover 11 and the second strap 13.

The straps 13 and 23 may surround a remaining portion that is not covered by the covers 11 and 21 and the fastening frames 12 and 22 in the circumference of the user's thighs and may include an elastic material (e.g., a band).

In an embodiment, the wearable device 100 may support a proximal part and a distal part of the user and may assist a relative motion between the proximal part and the distal part. Among the components of the wearable device 100, the components worn on the proximal part of the user may be referred to as a “proximal wearing part” and the components worn on the distal part of the user may be referred to as a “distal wearing part”. For example, among the components of the wearable device 100, the housing 80, the waist support frames 20 and 25, the pair of belts 60, and the auxiliary belt 70 may correspond to a proximal wearing part, and the thigh fastening portions 1 and 2 may correspond to a distal wearing part. For example, the proximal wearing part may be worn on the waist or pelvis of the user, and the distal wearing part may be worn on a thigh or a calf of the user. Positions in which the proximal wearing part and the distal wearing part are worn are not limited thereto. For example, the proximal wearing part may be worn on the torso or a shoulder of the user, and the distal wearing part may be worn on an upper arm or a lower arm of the user.

FIGS. 5A and 5B are diagrams illustrating a configuration of a control system of a wearable device according to an embodiment.

Referring to FIG. 5A, a wearable device (e.g., the wearable device 100) may be controlled by a control system 500. The control system 500 may include the control module 510, the sensor module 520, a driving module 530, and a battery 540. The driving module 530 may include a motor 534 to generate power (e.g., torque), and a motor driver circuit 532 to drive the motor 534. In the example of FIG. 5A, one sensor module 510, and the driving module 530 including one motor driver circuit 532 and one motor 534 are illustrated, however, this is merely an example. Referring to FIG. 5B, as shown in the illustrated example, a plurality of (e.g., two or more) sensor modules 520 and 520-1, motor driver circuits 532 and 532-1, and motors 534 and 534-1 may be provided. The driving module 530 including the motor driver circuit 532 and the motor 534 may correspond to the first driving module 45 of FIG. 3, and a driving module 530-1 including the motor driver circuit 532-1 and the motor 534-1 may correspond to the second driving module 35 of FIG. 3. The following description of each of the sensor module 520, the motor driver circuit 532, and the motor 534 may also be applied to the sensor module 520-1, the motor driver circuit 532-1, and the motor 534-1 shown in FIG. 5B.

Referring back to FIG. 5A, the sensor module 520 may include at least one sensor. The sensor module 520 may obtain sensor data that changes according to a motion of a user. The sensor module 520 may obtain acoustic signal data by converting vibrations generated by sound into electrical signals. The acoustic signal data may include, for example, voice signal data corresponding to human speech. The sensor module 520 may include, for example, an IMU (e.g., the IMU 135 of FIG. 1) to measure an upper body motion value of a user and an angle sensor (e.g., the angle sensor 125 of FIG. 1) to measure a hip joint angle value of the user, but is not limited thereto. For example, the sensor module 520 may further include at least one of a position sensor, a temperature sensor, a voice recognition sensor, a biosignal sensor, or a proximity sensor.

The sensor module 520 may transmit the obtained sensor data to the control module 510. The sensor module 520 may include, for example, an IMU, an angle sensor (e.g., an encoder or a hall sensor), a position sensor, a proximity sensor, a biosignal sensor, and a temperature sensor. The IMU may measure an upper body motion value of the user. For example, the IMU may sense the acceleration and angular velocity of an X-axis, a Y-axis, and a Z-axis according to a motion of the user. The angle sensor may measure a hip joint angle value according to a leg motion of the user. Sensor data that may be measured by the angle sensor may include, for example, a hip joint angle value of the right leg, a hip joint angle value of the left leg, and information on a direction of a motion of a leg.

The battery 540 may supply power to each component of the wearable device. The wearable device may convert the power of the battery 540 into power suitable for an operating voltage of each component of the wearable device and supply the converted power to each component.

The driving module 530 may generate an external force to be applied to a leg of the user under the control of the control module 510. The driving module 530 may be disposed in a position corresponding to a position of the hip joint of the user and may generate torque to be applied to a leg of the user based on a control signal generated by the control module 510. The control module 510 may transmit the control signal to the motor driver circuit 532, and the motor driver circuit 532 may generate a current signal corresponding to the control signal and supply the current signal to the motor 534, to control an operation of the motor 534. The current signal may not be supplied to the motor 534 according to the control signal. When the current signal is supplied to the motor 534, and the motor is driven, the motor 534 may generate force to assist a leg motion of the user or torque to impede the leg motion of the user.

The control module 510 may control an overall operation of the wearable device, and may generate a control signal to control each component (e.g., the driving module 530). The control module 510 may include a processor 512, a memory 514, and a communication module 516.

The processor 512 may execute, for example, software to control at least one other component (e.g., a hardware or software component) of the wearable device connected to the processor 512, and may perform a variety of data processing or computation. According to an embodiment, as at least a part of data processing or computation, the processor 512 may store instructions or data received from another component (e.g., the communication module 516) in the memory 514, process the instructions or the data stored in the memory 514, and store result data in the memory 514. According to an embodiment, the processor 512 may include a main processor (e.g., a central processing unit (CPU) or an application processor (AP)) or an auxiliary processor (e.g., a graphics processing unit (GPU), a neural processing unit (NPU), an image signal processor (ISP), a sensor hub processor, or a communication processor (CP)) that is operable independently of, or in conjunction with the main processor. The auxiliary processor may be implemented separately from the main processor or as a part of the main processor. Each “processor” herein includes processing circuitry, and/or may include multiple processors. For example, as used herein, including the claims, the term “processor” may include various processing circuitry, including at least one processor, wherein one or more of at least one processor, individually and/or collectively in a distributed manner, may be configured to perform various functions described herein. As used herein, when “a processor”, “at least one processor”, and “one or more processors” are described as being configured to perform numerous functions, these terms cover situations, for example and without limitation, in which one processor performs some of recited functions and another processor(s) performs other of recited functions, and also situations in which a single processor may perform all recited functions. Additionally, the at least one processor may include a combination of processors performing various of the recited/disclosed functions, e.g., in a distributed manner. At least one processor may execute program instructions to achieve or perform various functions.

The memory 514 may store a variety of data used by at least one component (e.g., the processor 512) of the control module 510. The variety of data may include, for example, software, sensor data, and input data or output data for instructions related thereto. The data may include, for example, computer-executable instructions. At least one of the instructions stored in the memory 514 may be executed by at least one processor 512 that may access the memory 514. The memory 514 may include a volatile memory or a non-volatile memory (e.g., random-access memory (RAM), dynamic RAM (DRAM), or static RAM (SRAM)).

The communication module 516 may support establishing a direct (e.g., wired) communication channel or a wireless communication channel between the control module 510 and another component of the wearable device or an external electronic device (e.g., the electronic device 210 or the server 230 of FIG. 2), and performing communication via the established communication channel. For example, the communication module 516 may transmit the sensor data obtained by the sensor module 520 to the external electronic device (e.g., the electronic device 210 of FIG. 2) and receive a control signal from the external electronic device. According to an embodiment, the communication module 516 may include one or more CPs that are operable independently of the processor 512 and that support direct (e.g., wired) communication or wireless communication. According to an embodiment, the communication module 516 may include a wireless communication module (e.g., a cellular communication module, a short-range wireless communication module, or a global navigation satellite system (GNSS) communication module), and/or a wired communication module. A corresponding one of these communication modules may communicate with another component of the wearable device and/or the external electronic device via a short-range communication network, such as Bluetooth™, wireless-fidelity (Wi-Fi), adaptive network topology (ANT) or infrared data association (IrDA), or a long-range communication network, such as a legacy cellular network, a 5G network, a next-generation communication network, the Internet, or a computer network (e.g., a local area network (LAN) or a wide region network (WAN)).

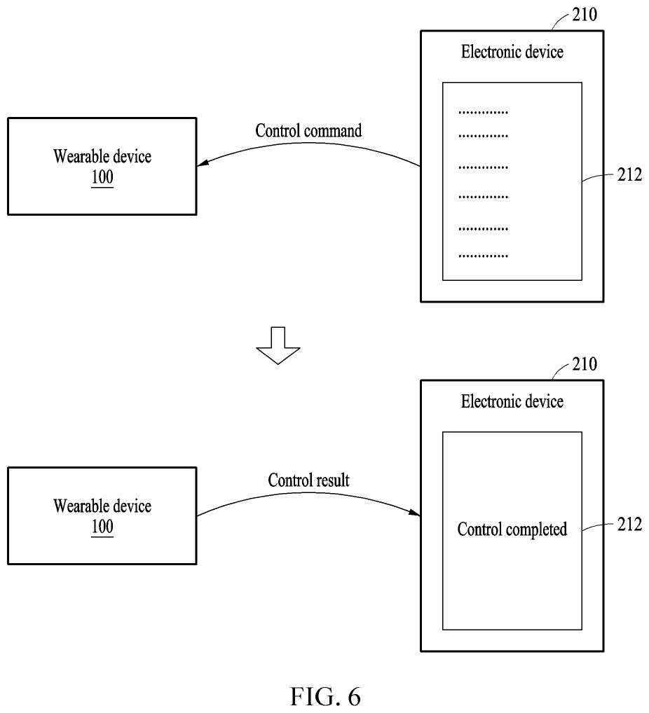

FIG. 6 is a diagram illustrating an interaction between a wearable device and an electronic device according to an embodiment.

Referring to FIG. 6, the wearable device 100 may communicate with the electronic device 210. For example, the electronic device 210 may be a user terminal of a user using the wearable device 100, or an exclusive controller for the wearable device 100. According to an embodiment, the wearable device 100 and the electronic device 210 may be connected using a short-range wireless communication scheme.

The electronic device 210 may control an operation of the wearable device 100 or display a user interface (UI) screen on a display 212. In an embodiment, the user may input a command to control the operation of the wearable device 100 through the UI screen on the display 212 of the electronic device 210. The electronic device 210 may generate a control command corresponding to the command and transmit the generated control command to the wearable device 100. The wearable device 100 may operate according to the received control command and transmit a control result and/or measured data (e.g., sensor data) to the electronic device 210. The electronic device 210 may provide the user with result information derived by analyzing the control result and/or data of the wearable device 100 through the display 212.

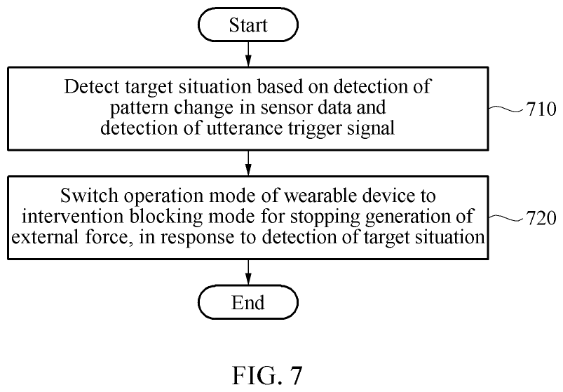

FIG. 7 is a flowchart illustrating an operation of a wearable device according to an embodiment.

Referring to FIG. 7, an operation method of a wearable device (e.g., the wearable device 100) according to an embodiment may include detecting 710 a target situation, based on the detection of a pattern change in sensor data and detection of an utterance trigger signal.

According to an embodiment, the sensor data may include motion data of a user (e.g., the user 110) wearing the wearable device collected from a sensor module (e.g., the sensor module 520) of the wearable device. For example, the motion data of the user may include sensor data obtained from an IMU (e.g., the IMU 135) of the wearable device. For example, the motion data of the user may include sensor data obtained from an angle sensor (e.g., the angle sensor 125) of the wearable device.

According to an embodiment, the sensor data may include data collected from a sensor (e.g., an IMU, an atmospheric pressure sensor, or a voice recognition sensor) of an electronic device (e.g., the electronic device 210) linked to the wearable device. For example, the sensor data may include data related to an image obtained from a camera of an electronic device linked to the wearable device. For example, the sensor data may include data related to an object around the user obtained from a TOF camera, Radar, or Lidar of an electronic device linked to the wearable device.

In an embodiment, the pattern change in the sensor data may include a pattern change in the sensor data related to a motion of a user wearing the wearable device. Hereinafter, a user may be a user wearing the wearable device. The sensor data related to a motion of the user may appear in a pattern corresponding to the user's motion type over time. For example, when the user is walking, the obtained sensor data may appear in a pattern corresponding to a motion type of walking. For example, when the user performs a predetermined type of exercise (e.g., squats), the obtained sensor data may appear in a pattern corresponding to the predetermined type of exercise.

The wearable device may detect a pattern change in the sensor data when a pattern of the sensor data related to the user's motion deviates from a normal pattern. For example, whether a pattern of the sensor data deviates from a normal pattern may be determined by comparing the pattern of the sensor data to a predetermined reference pattern. The operation method of the wearable device according to an embodiment may include detecting a pattern change in the sensor data based on a difference between a pattern of the sensor data and a reference pattern. The reference pattern may be determined based on an operation mode set in the wearable device. The operation mode of the wearable device may be set to a motion type that the user intends to perform. For example, when the user intends to perform walking, the operation mode of the wearable device may be set to a walking mode. For example, when the user intends to perform squats, the operation mode of the wearable device may be set to a squat mode. The reference pattern may correspond to a pattern of sensor data that typically appears when a motion corresponding to an operation mode of the wearable device is performed. The reference pattern corresponding to an operation mode supported by the wearable device may be stored in a memory (e.g., the memory 514) of the wearable device.

According to an embodiment, the wearable device may detect a pattern change in the sensor data when a difference between a pattern of the sensor data and the reference pattern exceeds a threshold level. There may be various methods for calculating a difference between a pattern of the sensor data and the reference pattern, and the threshold level may be determined according to the method for calculating the difference between the pattern of the sensor data and the reference pattern. For example, a difference between the pattern of the sensor data and the reference pattern may be calculated as a difference in factors that determine the form of a pattern, such as a difference between the amount of change in a value of the sensor data and the amount of change in a value of the reference pattern, a difference between the rate of change in a value of the sensor data and the rate of change in a value of the reference pattern, or a difference between the range of values included in the sensor data and the range of values included in the reference pattern.

For example, when the user performs a particular exercise motion (e.g., walking or running), the sensor data related to the user's motion may show consistent patterns over time. The reference pattern may correspond to a pattern of the sensor data in a section where a particular exercise motion is performed. When the user performs a particular exercise motion and then suddenly stops, the motion speed of the user may change drastically. In this case, sensor data related to acceleration obtained by measuring the user's motion from an IMU sensor of the wearable device may change rapidly in value at the moment the user suddenly stops. The sensor data of a section where the user suddenly stops may have a different pattern from the sensor data of the section where the user performs the particular exercise motion. The wearable device may detect a pattern change in the sensor data of the section where the user suddenly stops.

For example, when the user falls while performing a particular exercise motion, a height value of the wearable device may change rapidly due to the change in the user's posture. In this case, sensor data obtained from a height sensor of the wearable device may change rapidly at the moment the user falls. The sensor data of the section where the user falls may have a different pattern from the sensor data of the section where the user performs the particular exercise motion. The wearable device may detect a pattern change in the sensor data of the section where the user falls.

In addition, when an abnormal motion occurs that causes a large change in a value measured by the sensor of the wearable device, such as a sudden change in the inclination of the user's body while the user is performing a particular exercise motion, the wearable device may detect a pattern change in the sensor data of the section where the abnormal motion occurs.

In an embodiment, the utterance trigger signal may be a voice signal recognized as a scream. The voice signal of the user may be received from a voice recognition sensor of the wearable device or a voice recognition sensor of an electronic device linked to the wearable device.

According to an embodiment, the operation method of the wearable device may include detecting an utterance trigger signal based on a difference between waveform data of a received voice signal and reference waveform data. The waveform data of a voice signal may be a graph of the variation in air density over time due to a voice, and may appear in the form of a wave. In the waveform data of a voice signal, a greater amplitude of a wave may indicate a louder sound, and a shorter period of the wave (or a higher frequency) may indicate a higher-pitched sound. That is, data related to the size and pitch of a sound corresponding to a particular time may be obtained from the waveform data of the voice signal.

The wearable device may detect the utterance trigger signal when the waveform data of the voice signal deviates from the waveform data corresponding to normal speech. For example, whether the waveform data of the voice signal deviates from the waveform data corresponding to normal speech may be determined by comparing a pattern of the sensor data to predetermined reference waveform data. For example, the reference waveform data may be waveform data corresponding to an average size and pitch of people's voices. For example, the reference waveform data may be waveform data corresponding to an average size and pitch of a pre-registered user's voice.

According to an embodiment, the wearable device may detect the utterance trigger signal when a difference between the waveform data of a voice signal and the reference waveform data exceeds a threshold level. There may be various methods for calculating a difference between the waveform data of a voice signal and the reference waveform data, and the threshold level may be determined according to the method of calculating a difference between the waveform data of the voice signal and the reference waveform data. For example, a difference between the waveform data of the voice signal and the reference waveform data may be calculated as a difference in factors that determine the waveform data, such as a difference between the amplitude of the waveform data of a voice signal and the amplitude of the reference waveform data, or a difference between a wave period of the waveform data of the voice signal and a wave period of the reference waveform data.

According to an embodiment, the wearable device may detect a section of a voice signal where a difference occurs as an utterance trigger signal when a difference between the waveform data of the voice signal and the reference waveform data exceeds a threshold level for a threshold time or longer. For example, when, in the waveform data of a voice signal, a section in which the amplitude is greater than or equal to an average amplitude of the reference waveform data by a threshold value and the frequency is greater than or equal to an average frequency of the reference waveform data by the threshold value continues for a threshold time or longer, a signal in the corresponding section may be detected as an utterance trigger signal.

According to an embodiment, the operation method of the wearable device may include detecting an utterance trigger signal based on whether a recognition result of a received voice signal corresponds to a predetermined word. The recognition result of the voice signal may include text corresponding to the voice signal. For example, the recognition result of a voice signal that utters “Yikes” may be the text “Yikes”.

The recognition result of a voice signal may be obtained from a voice recognition model. The voice recognition model may include, for example, a neural network trained to perform voice recognition. The voice recognition model may be stored in the wearable device, an electronic device linked to the wearable device, or a server linked to the wearable device. When the voice recognition model is stored in an external device of the wearable device, the wearable device may obtain a voice recognition result for a voice signal from the external device. For example, the waveform data of a signal may be processed into data representing the amount of each frequency component included in a voice fragment (or frame) of a particular time length by Fourier transform. The voice signal processed by Fourier transform may be input to the voice recognition model.

The wearable device may detect the voice signal corresponding to the voice recognition result as an utterance trigger signal when the voice recognition result corresponds to a predetermined word. The predetermined word may include a predetermined word that may be recognized as a scream. For example, the predetermined word may include a word that a person utters when the person screams, such as “Yikes” or “Aargh”. The predetermined word may be stored in the wearable device, an electronic device linked to the wearable device, or a server linked to the wearable device. When the predetermined word is stored in an external device of the wearable device, the wearable device may obtain a result of whether the voice recognition result corresponds to the predetermined word from the external device.

A target situation may correspond to an event that occurs when both a pattern change in the sensor data and an utterance trigger signal are detected within a period of a predetermined length on the wearable device. That is, the target situation may be a situation in which a pattern of the sensor data related to a user's motion deviates from a normal pattern and a scream of the user is detected. For example, the target situation may be a hazardous situation in which a user wearing the wearable device experiences a motion that is different from a motion corresponding to an operation mode of the wearable device, such as falling or hitting a wall.

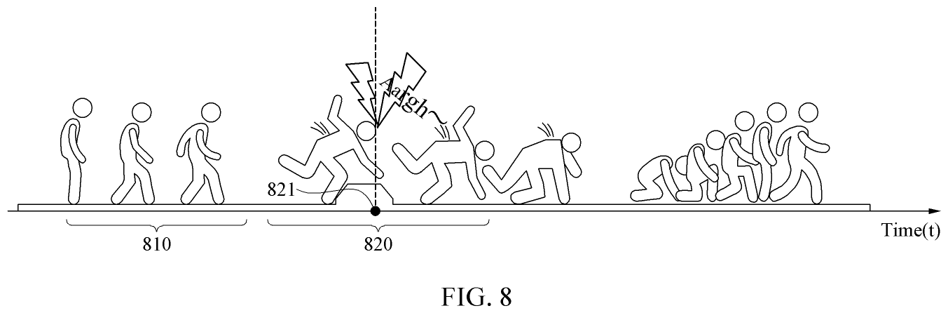

For example, FIG. 8 is a diagram illustrating a motion of a user (e.g., the user 110) wearing a wearable device (e.g., the wearable device 100) over time. Referring to section 810 of FIG. 8, the user may set an operation mode of the wearable device to a walking mode and perform a walking motion. Referring to section 820 of FIG. 8, the user may fall with a scream while performing a walking motion. In this case, a pattern of the sensor data related to the motion of the user may change rapidly as the user's motion changes rapidly. The wearable device may detect a pattern change in the sensor data of the section 820 where the user has fallen. In the section 820 where the user has fallen, a voice signal corresponding to waveform data different from the waveform data corresponding to a normal speech may be received due to the user's scream. The wearable device may detect an utterance trigger signal at the section 820 where the user has fallen. Since both a pattern change in the sensor data and an utterance trigger signal are detected in the section 820, the wearable device may detect that a target situation has occurred. According to an embodiment, a pattern change in the sensor data and an utterance trigger signal may be detected at a particular timepoint 821 within the section 820, and at the detected timepoint 821, the wearable device may detect the target situation.

According to an embodiment, the operation method of the wearable device may include switching an operation mode of the wearable device to an intervention blocking mode to stop the generation of external force, in response to the detection of the target situation. The intervention blocking mode may be an operation mode that prevents or reduces a chance of the wearable device from interfering with the user's motion, for example, a mode in which the wearable device stops the generation of external force applied to the user. For example, referring to FIG. 8, at the timepoint 821 when the target situation is detected, the wearable device may switch the operation mode to the intervention blocking mode. When switched to the intervention blocking mode, the wearable device may stop the generation of external force applied to the user. Switching to the intervention blocking mode may result in the wearable device no longer applying external force to the user. The wearable device may reduce the possibility of injury to the user by stopping the application of external force to the user when the target situation is detected due to an abnormal motion (e.g., a sudden stop, fall, etc.) of the user, thereby allowing the user to freely control their body without hindering the user's motion.

According to an embodiment, switching 720 to the intervention blocking mode may include switching the operation mode of the wearable device to the intervention blocking mode based on a setting for whether to use the intervention blocking mode when the target situation is detected. The user may determine a setting value for whether to use the intervention blocking mode of the wearable device. For example, the setting value for whether to use the intervention blocking mode may be input through a program for controlling the wearable device installed on an electronic device. When set to use the intervention blocking mode, in response to the detection of a target situation, the operation mode of the wearable device may be switched to the intervention blocking mode.

When set not to use the intervention blocking mode, in response to the detection of a target situation, the operation mode of the wearable device may not be switched to the intervention blocking mode. In this case, an external force corresponding to the operation mode may be continuously generated in the wearable device. For example, when the operation mode of the wearable device is a walking assistance mode, external force may be continuously generated by the wearable device to assist the user's walking, and the external force generated by the wearable device may continue to be applied to the user.

According to an embodiment, the external force corresponding to the operation mode may include an external force having a predetermined direction and intensity corresponding to the operation mode. The external forces generated in different operation modes may differ in at least one of a direction and intensity. For example, in an operation mode of the walking assistance mode, the wearable device may generate an external force of a direction and intensity corresponding to the walking assistance mode. For example, in an operation mode of an exercise assistance mode, the wearable device may generate an external force of a direction and intensity corresponding to the exercise assistance mode. The external force generated in response to the walking assistance mode and the external force generated in response to the exercise assistance mode may differ from each other in at least one of a direction and intensity. For example, the exercise assistance mode may be divided into detailed operation modes depending on the type of exercise. For example, the exercise assistance mode may include at least one of a squat mode, a running mode, and a walking mode. According to an embodiment, the predetermined direction and intensity of an external force corresponding to the operation mode may be corrected based on the user's characteristics.

According to an embodiment, the switching 720 to the intervention blocking mode may include controlling a driving module (e.g., the driving module 120 of FIG. 1, the driving modules 35 and 45 of FIG. 3, the driving module 45 of FIG. 4, the driving module 530 of FIG. 5A, the driving module 530 of FIG. 5B, or the driving module 530-1 of FIG. 5B) related to the generation of external force of the wearable device such that the external force is not generated, based on a setting related to the intervention blocking mode. The switching 720 to the intervention blocking mode may include controlling the driving module such that the external force is gradually reduced based on the setting related to the intervention blocking mode. For example, the setting related to the intervention blocking mode may include a setting related to a manner in which the generation of the external force is stopped in the intervention blocking mode. Depending on a setting value of the manner in which the generation of external force is stopped, the external force generated by the wearable device may be stopped immediately. Depending on a setting value of the manner in which the generation of external force is stopped, the external force generated by the wearable device may be stopped gradually. For example, the manner in which the generation of external force is stopped may be set so that the intensity of the external force is reduced by a certain amount at a certain period.

According to an embodiment, the operation method of the wearable device may include outputting a signal to indicate that an operation mode has been switched to the intervention blocking mode. A signal to indicate that the operation mode has been switched to the intervention blocking mode may be output via an output device of the wearable device and/or an output device of an electronic device linked to the wearable device in a manner perceptible to the user. For example, the signal to indicate that the operation mode has been switched to the intervention blocking mode may be output in an audible form via a speaker of the wearable device and/or a speaker of an electronic device linked to the wearable device. For example, the signal to indicate that the operation mode has been switched to the intervention blocking mode may be output in a visual form via a display of the wearable device and/or a display (e.g., the display 212) of an electronic device linked to the wearable device. For example, the signal to indicate that the operation mode has been switched to the intervention blocking mode may be output in the form of a vibration.

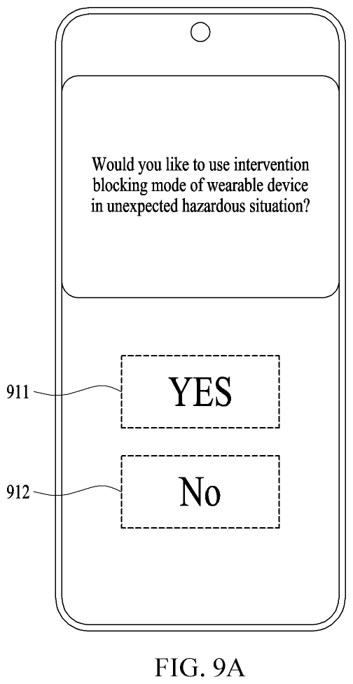

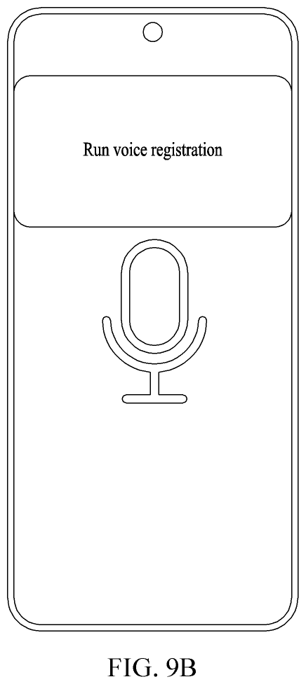

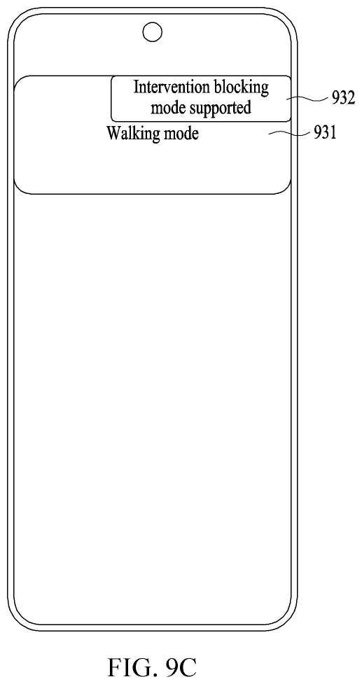

FIGS. 9A to 9C are diagrams illustrating UI screens for setting an intervention blocking mode of a program for controlling a wearable device according to an embodiment.

For example, a program for controlling a wearable device (e.g., the wearable device 100) may be installed in a user terminal (e.g., the user terminal 220) linked to the wearable device. A user (e.g., the user 110) may execute the program installed on the user terminal and input a setting value for the wearable device through the program.

Referring to FIG. 9A, a UI screen for setting the intervention blocking mode through the program may be provided through a display (e.g., the display 212) of the user terminal. The user may input a setting value to use the intervention blocking mode of the wearable device by selecting a button 911. The user may input a setting value to not use the intervention blocking mode of the wearable device by selecting a button 912.

Referring to FIG. 9B, a UI screen for registering a user's voice through the program may be provided through the display (e.g., the display 212) of the user terminal. The user may register their voice through an input device of the user terminal. Reference waveform data for detecting an utterance trigger signal may be generated based on registered voice data. The reference waveform data may include data such as an average size and pitch of a registered voice of the user.

Referring to FIG. 9C, a UI screen that indicates an operation mode of the wearable device through a program may be provided through the display (e.g., the display 212) of the user terminal. The user may identify the operation mode (e.g., a walking mode) of the wearable device through the program. For example, the UI screen may include an indication 931 of an operation mode that indicates the operation mode of the wearable device. The user may change the operation mode of the wearable device to another mode through the program. When set to use the intervention blocking mode, the operation mode of the wearable device may be switched to the intervention blocking mode in response to a target situation being detected. When the operation mode of the wearable device is changed, the changed operation mode may be indicated through the UI screen.

The user may identify a setting value for whether to use the intervention blocking mode of the wearable device through the program. For example, when set to the intervention blocking mode, an indication 932 to indicate whether the intervention blocking mode is supported may be additionally displayed along with the indication 931 of the operation mode of the wearable device on the UI screen that indicates the operation mode of the wearable device.

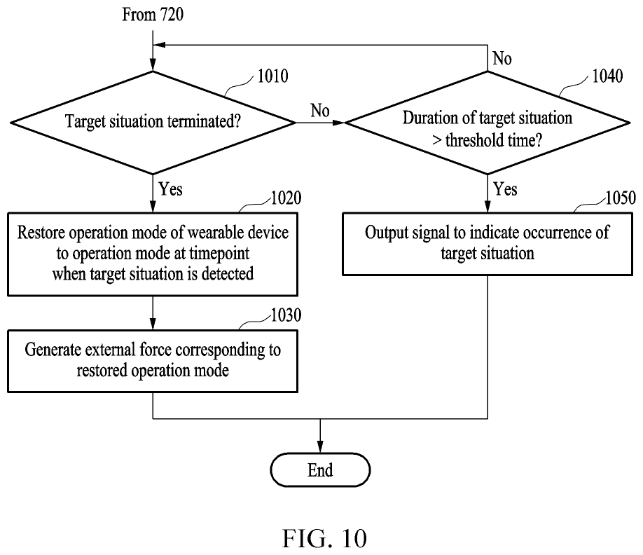

FIG. 10 is a flowchart illustrating an operation of a wearable device corresponding to whether a target situation has been terminated after switching to an intervention blocking mode, according to an embodiment.

Referring to FIG. 10, an operation method of a wearable device (e.g., the wearable device 100) according to an embodiment may include determining 1010 whether a target situation has been terminated. For example, the wearable device may detect the termination of a target situation based on the detection of a pattern of sensor data corresponding to a reference pattern. The pattern of the sensor data corresponding to a reference pattern may be a pattern of sensor data that generally appears when a motion corresponding to an operation mode at a timepoint when the target situation of the wearable device is detected is performed. When the pattern of the sensor data corresponding to the reference pattern is detected, the target situation may be determined to be terminated.

The operation method of the wearable device according to an embodiment may include restoring 1020 the operation mode of the wearable device to the operation mode at the timepoint when the target situation is detected, in response to the termination of the target situation. The operation mode of the wearable device may be switched from the intervention blocking mode to the operation mode at the timepoint when the target situation is detected. For example, when the operation mode of the wearable device is switched from a first mode to the intervention blocking mode in response to the detection of the target situation, the operation mode of the wearable device may be switched from the intervention blocking mode to the first mode in response to the termination of the target situation.

The operation mode of the wearable device according to an embodiment may include generating 1030 an external force corresponding to the restored operation mode. For example, when the restored operation mode of the wearable device is a walking assistance mode, an external force may be generated in the wearable device to assist the walking of a user (e.g., the user 110). For example, when the restored operation mode of the wearable device is an exercise assistance mode, an external force may be generated in the wearable device to assist the exercise of the user.

The operation method of the wearable device according to an embodiment may include determining 1040 whether a duration of the target situation exceeds a threshold time when the termination of the target situation is not detected. The operation method of the wearable device may include outputting 1050 a signal to indicate the occurrence of the target situation when the target situation continues for a threshold time or longer. The signal to indicate the occurrence of the target situation may be output via an output device of the wearable device and/or an output device of an electronic device (e.g., the electronic device 210) linked to the wearable device in a manner perceptible to the user. For example, the signal to indicate the occurrence of the target situation may be output in an audible form via a speaker of the wearable device and/or a speaker of an electronic device linked to the wearable device. For example, the signal to indicate the occurrence of the target situation may be output in a visual form via a display of the wearable device and/or a display (e.g., the display 212) of an electronic device linked to the wearable device. For example, the signal to indicate the occurrence of the target situation may be output in the form of a vibration.

According to an embodiment, the signal to indicate the occurrence of the target situation may be transmitted to another device. For example, the wearable device may transmit an SOS signal to a number designated as an emergency contact. For example, a user terminal linked to the wearable device may transmit an SOS signal to a number designated as an emergency contact.

The wearable device according to an embodiment may include a driving module (e.g., the driving module 120 of FIG. 1, the driving modules 35 and 45 of FIG. 3, the driving module 35 of FIG. 4, the driving module 530 of FIG. 5A, the driving module 530 of FIG. 5B, or the driving module 530-1 of FIG. 5B) configured to generate an external force corresponding to an operation mode. The wearable device may include a memory (e.g., the memory 514) in which computer-executable instructions are stored. The wearable device may include a processor (e.g., the processor 512) configured to execute the instructions by accessing the memory. The instructions may be configured to cause the processor to detect a target situation, based on the detection of a pattern change in sensor data and the detection of an utterance trigger signal. The instructions may be configured to cause the processor to switch an operation mode of the wearable device to an intervention blocking mode to stop the generation of external force, in response to the detection of the target situation.

The wearable device according to an embodiment may include a sensor module (e.g., the sensor module 520) configured to obtain sensor data including motion data of a user wearing the wearable device.

The wearable device according to an embodiment may include a communication module (e.g., the communication module 516) configured to receive sensor data from at least one electronic device linked to the wearable device.

The instructions according to an embodiment may be configured to cause the processor to detect a pattern change in the sensor data, based on a difference between a pattern of the sensor data and a reference pattern. The reference pattern may be determined based on an operation mode set in the wearable device.

The instructions according to an embodiment may be configured to cause the processor to detect an utterance trigger signal based on a difference between waveform data of a received voice signal and reference waveform data. The reference waveform data may be determined based on a waveform of a speech voice of a human.

The instructions according to an embodiment may be configured to cause the processor to detect the utterance trigger signal based on whether a recognition result of the received voice signal corresponds to a predetermined word.

The instructions according to an embodiment may be configured to cause the processor to control the driving module such that external force is not generated, based on a setting of the intervention blocking mode.

The instructions according to an embodiment may be configured to cause the processor to control the driving module such that the external force is gradually reduced, based on a setting of the intervention blocking mode.

The instructions according to an embodiment may be configured to cause the processor to detect the termination of a target situation based on the detection of a pattern of sensor data corresponding to a reference pattern. The instructions according to an embodiment may be configured to cause the processor to, in response to the termination of the target situation, restore the operation mode of the wearable device to an operation mode prior to the detection of the target situation. The instructions according to an embodiment may be configured to cause the processor to generate an external force corresponding to the restored operation mode. “Based on” as used herein covers based at least on.

The instructions according to an embodiment may be configured to cause the processor to output a signal to indicate that the operation mode has been switched to the intervention blocking mode.

The instructions according to an embodiment may be configured to cause the processor to output a signal to indicate the occurrence of the target situation when the target situation continues for a threshold time or longer.

According to embodiments of the present disclosure, when an abnormal motion (e.g., a sudden stop, fall, etc.) of a user occurs, the wearable device may stop the application of external force to the user, thereby reducing the possibility of injury and/or the extent of injury to the user by allowing the user to freely control their body without hindering the user's motions.

It should be appreciated that various embodiments of the present disclosure and the terms used therein are not intended to limit the technological features set forth herein to particular embodiments and include various changes, equivalents, or replacements for a corresponding embodiment. In connection with the description of the drawings, like reference numerals may be used for similar or related components. It is to be understood that a singular form of a noun corresponding to an item may include one or more of the things, unless the relevant context clearly indicates otherwise. As used herein, “A or B,” “at least one of A and B,” “at least one of A or B,” “A, B or C,” “at least one of A, B and C,” and “at least one of A, B, or C,” may include any one of the items listed together in the corresponding one of the phrases, or all possible combinations thereof. Terms such as “1st” and “2nd,” or “first” and “second” may be used to simply distinguish a corresponding component from other components, and do not limit the components in other aspects (e.g., importance or order). It is to be understood that if an element (e.g., a first element) is referred to, with or without the term “operatively” or “communicatively,” as “coupled with,” “coupled to,” “connected with,” or “connected to” another element (e.g., a second element), the element may be coupled with the other element directly (e.g., by wire), wirelessly, or via at least a third element(s). Thus, for example, “connected” as used herein covers both direct and indirect connections.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC). Thus, each “module” herein may comprise circuitry.

The software may include a computer program, a piece of code, an instruction, or some combination thereof, to independently or collectively instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums. Embodiments as set forth herein may be implemented as software including one or more instructions that are stored in a storage medium (e.g., the memory 514) that is readable by a machine. For example, a processor of the machine may invoke at least one of the one or more instructions stored in the storage medium and execute it. This allows the machine to be operated to perform at least one function according to the at least one instruction invoked. The one or more instructions may include code generated by a compiler or code executable by an interpreter. The machine-readable storage medium may be provided in the form of a non-transitory storage medium. Here, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to embodiments of the disclosure may be included and provided in a computer program product. The computer program product may be traded as a product between a seller and a buyer. The computer program product may be distributed in the form of a machine-readable storage medium (e.g., compact disc read-only memory (CD-ROM)), or be distributed (e.g., downloaded or uploaded) online via an application store (e.g., PlayStore™), or between two user devices (e.g., smartphones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components or operations may be omitted, or one or more other components or operations may be added. Alternatively or additionally, a plurality of components (e.g., modules or programs) may be integrated into a single component. In such a case, the integrated component may still perform one or more functions of each of the plurality of components in the same or similar manner as they are performed by a corresponding one of the plurality of components before the integration. According to embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various embodiments, it will be understood that the various embodiments are intended to be illustrative, not limiting. It will further be understood by those skilled in the art that various changes in form and detail may be made without departing from the true spirit and full scope of the disclosure, including the appended claims and their equivalents. It will also be understood that any of the embodiment(s) described herein may be used in conjunction with any other embodiment(s) described herein.

1. A wearable device, comprising:
a driving module, comprising a motor and/or circuitry, configured to generate an external force corresponding to an operation mode;
a memory configured to store computer-executable instructions; and
at least one processor, comprising processing circuitry, configured to individually and/or collectively execute the instructions by accessing the memory,
wherein, when executed by the at least one processor, the instructions are configured to cause the processor to:
detect a target situation, based on detection of a pattern change in sensor data and detection of an utterance trigger signal, and
in response to the detection of the target situation, switch an operation mode of the wearable device to an intervention blocking mode for stopping generation of the external force.

2. The wearable device of claim 1, further comprising:
a sensor module, comprising at least one sensor, configured to obtain sensor data comprising motion data of a user of the wearable device.

3. The wearable device of claim 1, further comprising:
a communication module, comprising communication circuitry, configured to receive the sensor data from at least one electronic device linked to the wearable device.

4. The wearable device of claim 1, wherein,
the instructions are further configured to cause the at least one processor to, detect the pattern change in the sensor data, based on a difference between a pattern of the sensor data and a reference pattern,
wherein the reference pattern is determined based on an operation mode set for the wearable device.

5. The wearable device of claim 1, wherein,
the instructions are further configured to cause the at least one processor to, detect the utterance trigger signal based on a difference between waveform data of a received voice signal and reference waveform data,
wherein the reference waveform data is determined based on a waveform of a speech voice of a human.

6. The wearable device of claim 1, wherein,
the instructions are further configured to cause the at least one processor to, detect the utterance trigger signal based on whether a recognition result of the received voice signal corresponds to a predetermined word.

7. The wearable device of claim 1, wherein,
the instructions are further configured to cause the at least one processor to, based on a setting of the intervention blocking mode, control the driving module such that the external force is not generated.

8. The wearable device of claim 1, wherein,
the instructions are further configured to cause the at least one processor to, based on the setting of the intervention blocking mode, control the driving module such that the external force is gradually reduced.

9. The wearable device of claim 1, wherein,
the instructions are further configured to cause the at least one processor to, detect a termination of the target situation, based on the detection of a pattern of the sensor data corresponding to a reference pattern,
in response to the termination of the target situation, restore the operation mode of the wearable device to an operation mode prior to the detection of the target situation, and
generate an external force corresponding to the restored operation mode.

10. The wearable device of claim 1, wherein,
the instructions are further configured to cause the at least one processor to, output a signal to indicate that the operation mode has been switched to the intervention blocking mode.

11. The wearable electronic device of claim 1, wherein,
the instructions are further configured to cause the at least one processor to, in response to the target situation continuing for a threshold time or longer, output a signal to indicate the occurrence of the target situation.

12. An operation method of a wearable device, the operation method comprising:
detecting a target situation, based on detection of a pattern change in sensor data and detection of an utterance trigger signal; and
in response to the detection of the target situation, switching an operation mode of the wearable device to an intervention blocking mode for stopping generation of an external force.

13. The operation method of claim 12, further comprising:
detecting the pattern change in the sensor data, based on a difference between a pattern of the sensor data and a reference pattern,
wherein the reference pattern is determined based on an operation mode set for the wearable device.

14. The operation method of claim 12, further comprising at least one of:
detecting the utterance trigger signal based on a difference between waveform data of a received voice signal and reference waveform data; and
detecting the utterance trigger signal based on whether a recognition result of the received voice signal corresponds to a predetermined word.

15. A non-transitory computer-readable storage medium storing instructions that, when executed by a processor, cause the processor to perform the operation method of claim 12.
